(19) United States
(12) Patent Application Publication
(10) Pub. No.: US 2003/0040701 A1
(43) Pub. Date: Feb. 27, 2003

(54) Dual chamber syringe with a dual function piston

(76) Inventor: Asger Lau Dalmose, Aabyhoj (DK)

Correspondence Address: James C Wray, Suite 300, 1493 Chain Bridge Road, McLean, VA, 22101, US

(21) Appl. No.: 10/169,254

(22) PCT Filed: Dec. 28, 2000
(86) PCT No.: PCT/DK00/00736

(30) Foreign Application Priority Data
Dec. 28, 1999 (DK) PA 1999 01862

(51) Int. Cl. A61M 37/00

(52) U.S. Cl. 604/87; 604/85; 604/191

(57) ABSTRACT

A dual chamber syringe in which a dual function piston divides the syringe into two compartments containing powder or fluid in one compartment and fluid in the other. For mixture of the two substances, a passage is opened between the two compartments before or during retraction of the piston to force the substances to be mixed in the front compartment. During forward movement of the piston, the passage between the two compartments is closed to force the mixture of substances through the discharge opening of the syringe.

The present invention relates to a dual chamber syringe where a dual function piston divides the barrel of the syringe into two compartments.

In many cases, medicine may be stored for longer time in powder form than in liquid form. When using the traditional preparation technique, liquid is aspirated from one container into a syringe and from this inserted into a powder container to mix with the powder, after which the resulting solution is aspirated into the syringe for later injection. In order to reduce the amount of objects necessary for preparation of the medicine and reduce the risk for errors and injury, syringes with two compartments, so-called dual chamber syringes, have been developed

From U.S. Pat. No. 4,563,174, a dual chamber syringe is known, where the two chambers contain two different components to be mixed prior to injection of the components. A barrel containing a first component, preferably a powder, comprises an end with a needle receiving nipple opposite to a wide open end to receive an assembly of a plug and a plunger. The plunger has a constant volume for containing a second component, preferably a liquid. During storage, the plunger is sealed by a part of the plug extending into the plunger. For admixture of the liquid with the powder, the plunger is turned relative to the plug whereby narrow channels are opened between the volume inside the barrel and the volume inside the plunger for mixture of the liquid and the powder. Filling of the syringe is preferably done by filling liquid into the plunger, closing the plunger with the plug and then inserting the plug/plunger assembly into the barrel, which has been sealed with the nipple beforehand.

This syringe has a number of disadvantages. When the channels are opened in order to mix liquid and powder, only gravity can force the liquid through the channels and into the barrel. Because the volume inside the plunger is constant, air has to leave the barrel and flow into the plunger at the same time as liquid enters the barrel. However, this is extremely difficult to achieve, which will be explained in the following. When orientating the syringe such that the nipple is downwards, the channels are filled with liquid preventing air to flow from the barrel volume into the plunger volume. In practice, it is almost impossible to mix the two components. The functioning is especially difficult in the first of the disclosed embodiments in the patent due to the following reason. When the plunger is turned in order to screw the plunger from the conical part of the plug, the volume in the plug/plunger assembly is increased resulting in a lower pressure in this plug/plunger assembly. This might result in powder being sucked into the small channels where a mixture of this powder in the channel and the liquid in the channel easily causes clogging in the channels and malfunctioning of the syringe. Furthermore, the described preferred filling method is disadvantageous. When the plug/plunger assembly is inserted into the nipple-sealed barrel, air is compressed inside the barrel causing the pressure in the barrel to be higher than in the plunger. When the channels between the plunger and the barrel are opened, air and powder is forced from the barrel volume into the channels causing clogging in the channels. Additionally, the pressure in the communicating barrel and plunger volume will still be higher than atmospheric pressure, which may cause air, liquid and powder to be pressed out of the nipple when the seal is broken Considering the fact, that the liquid and powder may contain cytotoxin, this implies a dangerous situation for the medical personal using the syringe.

From U.S. Pat. No. 4,693,706, a different dual chamber syringe is known. This syringe comprises an outer barrel, into which a first plunger is slidably inserted, this first plunger constituting a second barrel for a second plunger slidably inserted therein. The volume of the first barrel and the volume of the second barrel can be forced to communicate upon rupture or fracture of a membrane separating the two volumes.

This syringe has the severe disadvantage that small pieces from the fracture of the membrane accidentally may be injected into the patient and cause emboli.

From U.S. Pat. No. 5,779,668 a further dual chamber syringe is known. Also this syringe comprises an outer barrel, into which a first plunger is slidably inserted, this first plunger constituting a second barrel for a second plunger slidably inserted therein. The volume of the first and the second barrel can be forced to communicate in order to mix the liquid and the powder. In several embodiments of the invention, the volume of the first barrel and the volume of the second barrel can be forced to communicate after penetration of the reciprocable stopper with a cannula When penetrating the stopper, the cannula may punch a piece from the stopper, which finally may be transferred to a patient by injection and cause emboli. Generally, this system has the disadvantage of being complicated in construction, which results in very high production costs.

Several patents, for example U.S. Pat. No. 4,226,236, No. 4,613,236, No. 4,792,329, No. 5,788,670, No. 5,971,953 and European patent application EP 520 618 disclose dual chamber syringes, where the outer barrel is not cylindrical but comprises a broader region as a by-pass channel between two cylindrical compartments, one containing a first liquid and the other containing powder. A plunger separates these two compartments as long as the plunger is located in the cylindrical part of the barrel. When the plunger is moved into the broader region, the two compartments are connected through the by-pass channel for mixing of the liquid with the powder. This seemingly simple construction, however, has a disadvantage, as it is difficult and expensive to produce outer barrels with these by-pass channels. Furthermore, several of these dual chamber syringes are difficult to handle for proper mixture of liquid and powder.

It is an object of the present invention to provide a simple and reliable dual chamber syringe that is easy to use and easy and cheap to manufacture. Furthermore, it is an object of the invention that the syringe shall not cause any risk of emboli by fragments from a stopper, a membrane or from clogged powder.

This object is achieved with a dual chamber syringe comprising a hollow tubular barrel having a discharge opening at one end and a closure member at the opposite end. A piston that is slidably fitted into said barrel is connected to one end of a piston rod protruding through a sealed, fluid tight opening in said closure member. The volume between said piston and said discharge opening end constitutes a first chamber for a first substance and the volume between said piston and said closure member constitutes a second chamber for a second substance. Said piston has means for opening a passage between said first and said second chamber for forcing said second substance to flow from said second chamber into said first chamber during retraction of said piston, and said piston has means for closing said passage between said first and said second chamber for forcing said substance in said first chamber towards said discharge opening by forward movement of said piston Said closure member has means for opening an air inlet through said closure member for air supply to said second chamber during forward movement of said piston.

The sum of said first and second chamber can be assumed to be approximately constant, if the diameter of the piston rod is much smaller than the diameter of the second chamber, because the volume of the rod in this case is negligible.

In one embodiment of the invention, said piston comprises a throttle valve operable to open and close said passage by a turning of said piston rod. Preferably, said hollow tubular barrel has an oval cross section to prevent turning of at least one part of the piston, when the piston rod is turned while another part can rotate inside the barrel. As an alternative to the oval cross section, a polygonal cross section is possible.

Alternatively, said hollow tubular barrel may have a circular cross section with an axially extending inner rib mating with a complementary groove in the outer surface of at least one part of said piston in order to prevent that part of said piston to turn relative to said barrel, while another part can rotate inside the barrel.

In another embodiment of the invention, said piston comprises a valve with a flexible component having a cross section equal to the inner cross section of said barrel, wherein said flexible component by retraction of said piston is deformable such as to allow fluid from said second chamber to flow to said first chamber. The flexible component is, for example, a flexible membrane or a flexible ring on a rigid disc.

In a further embodiment of the invention, said piston comprises a bell shaped flexible member with a concave side towards said first chamber and a convex side towards said second chamber, said bell shaped flexible member having a peripheral hp arranged such as to be pressed against the inner surface of said barrel when the pressure in said first chamber is higher than in said second chamber, preferably during forward motion of said piston, in order to seal between the first and the second chamber, and arranged such as to be bend inwards when the pressure in said first chamber is lower than in said second chamber, preferably during retraction of said piston, in order to achieve said opening of said passage between said first and said second chamber. The principle of the piston and its function is known from bicycle air pumps.

The discharge opening can be constructed dependent on actual needs, and different principles are known and described in the literature. Preferentially, the discharge opening comprises a luer lock.

In order to prevent clogging of substance in the discharge opening prior to injection, the discharge opening can be occluded with a closure element that comprises a stem extending into said discharge opening for prevention of clogging of substance in the discharge opening. The closure element is removed before mount of a cannula or an intra venous access on the discharge opening

During storage and transport of the syringe according to the invention, it is important, that the piston is not activated accidentally, because that might cause unintended mixing of the substances. To prevent such unintended activation, the piston rod is connected to said barrel by a removable connector. The connector is constructed such that it is easily removable for intended use of the syringe.

The syringe according to the invention is a system, which is simple and reliable. It is easy and cheap to fabricate, especially because all components can be made of polymer, which also allows recycling of the material. As there are not created any fragments in the syringe, risk of emboli due to injected fragments is minimised. Also clogging in the discharge opening prior to injection is prevented according to a further embodiment of the invention, which reduces risk for emboli even further. Therefore, the syringe according to the invention is a very safe system in many ways superior to prior art. The syringe according to the invention has a further advantage superior to prior art syringes. Because the second substance from the second chamber due to the retraction of the piston is forced into the first chamber, it is possible to use the system also with very viscous fluids, which opens the possibilities for using such type of syringes in a much wider field than possible hitherto.

Furthermore, the syringe according to the invention is easy to fill. A substance, preferably a fluid, is filled through the air inlet in the closure member. In order to ensure easy filling of the second chamber, the closure member is equipped with two channels allowing a substance to be filled into the second chamber through one of the channels while air from the second chamber during filling can escape through the second channel. Another substance, for example powder or fluid, is filled into the first chamber through the discharge opening, after which that opening is sealed, for example by use of the earlier mentioned stem closure element Having a pressure in the first chamber slightly higher than in the second chamber, will increase the sealing pressure of the syringes with a valve comprising a flexible component.

The invention will be explained further in the following with reference to the drawings where

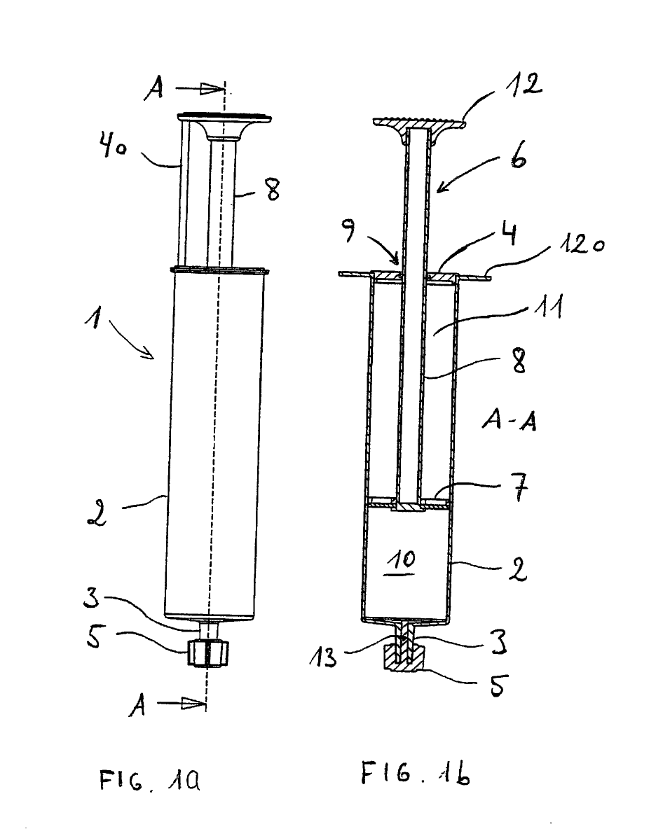

FIG. 1 is a schematic drawing of the dual chamber syringe according to the invention,

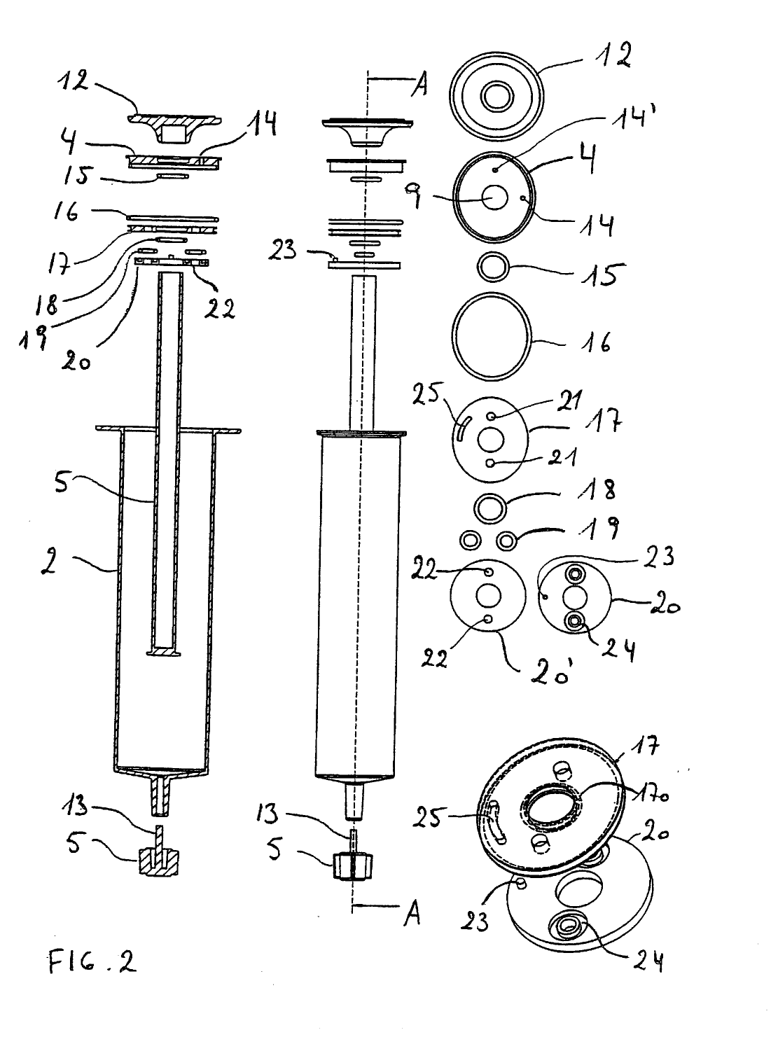

FIG. 2 is a schematic drawing of a dual chamber syringe according to the invention with a throttle valve piston,

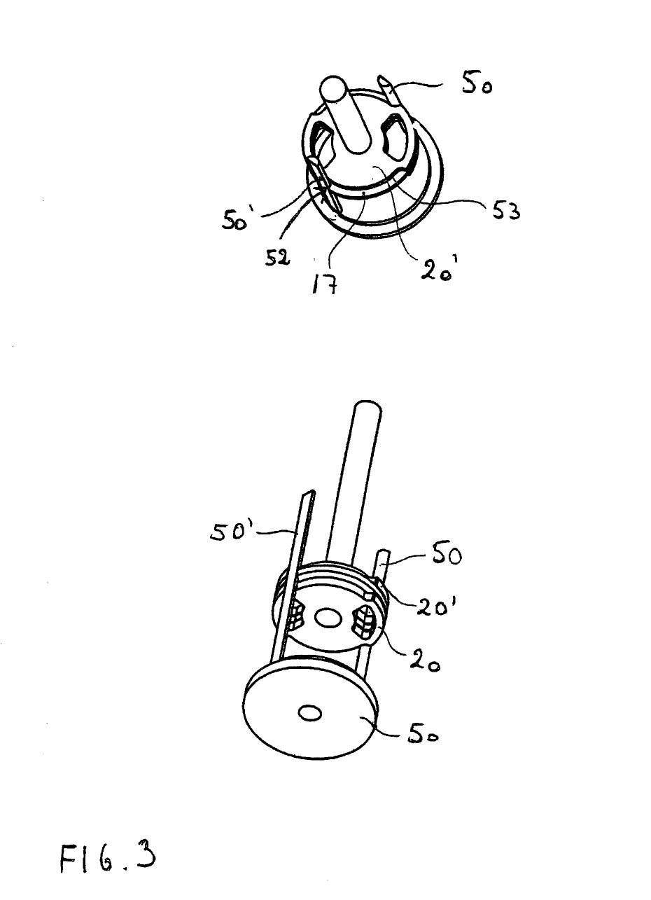

FIG. 3 is a schematic drawing of a throttle valve piston where turn of one component of the piston is prevented by an axially extending rib in the barrel,

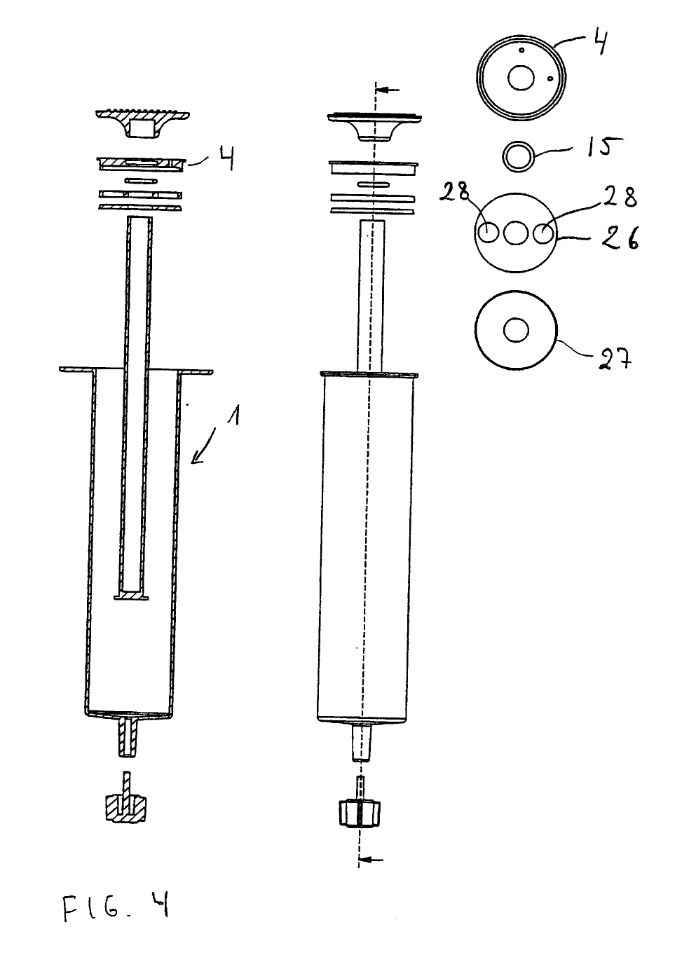

FIG. 4 is a schematic drawing of a dual chamber syringe according to the invention with a membrane valve,

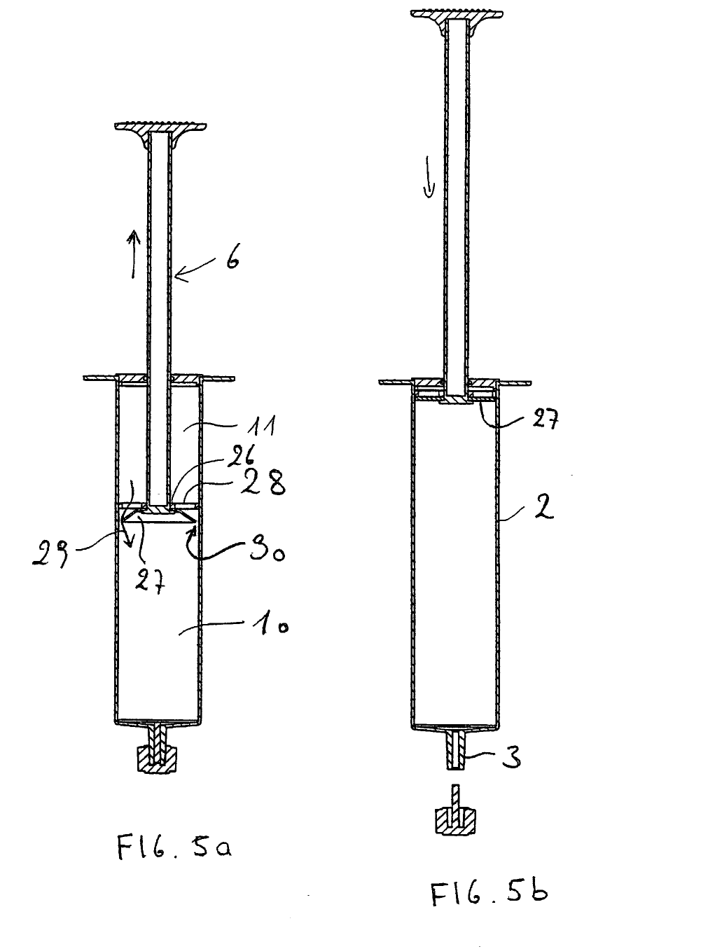

FIG. 5 shows the syringe with a membrane valve during and after retraction of the piston,

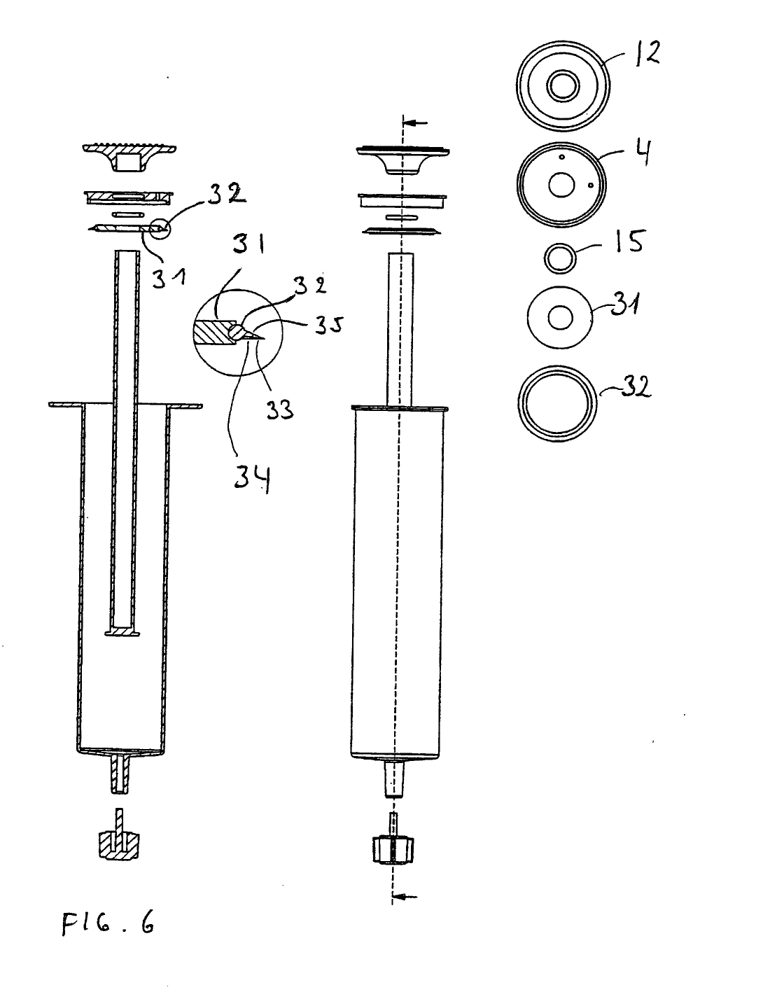

FIG. 6 is a schematic drawing of a dual chamber syringe according to the invention with a valve comprising a rigid disc with a flexible lip,

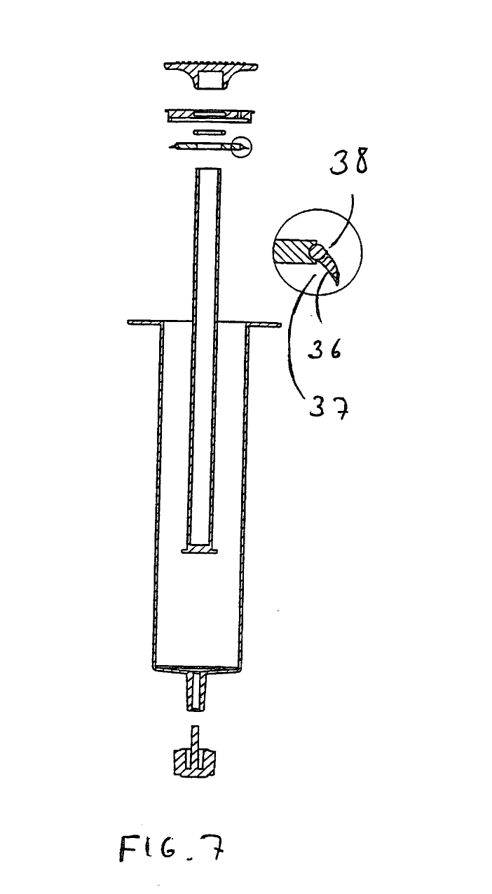

FIG. 7 is a schematic drawing of a dual chamber syringe according to the invention with a valve comprising a rigid disc with a flexible lip extending forwardly.

FIG. 1 is a schematic drawing of a dual chamber syringe 1 according to the invention, where FIG. 1a is a side view of the syringe 1 and FIG. 1b is a cross section of the syringe across the line A-A. The dual chamber syringe 1 comprises a hollow tubular barrel 2 having a discharge opening 3 at one end of the barrel 2 and a closure member 4 at the opposite end. The discharge opening 3 is occluded by a closure element 5. A piston rod 8 connected to a piston 7 is protruding through a sealed, fluid tight opening 9 in said closure member 4. The piston 7 is slidably fitted into said barrel 2. The total volume of the first chamber 10, intended for storage of a first substance, preferably powder, and the second chamber 11, intended for storage of a second substance, preferably a liquid, in the barrel is constant apart from the volume of the piston rod 8 inside the second chamber 11. However, the piston rod can be designed rather narrow such that its contribution to the volume is negligible. For movement of the piston 7, the piston rod 8 is equipped with a finger grip 12.

The finger grip 12 may be replaced with or connected to any other mechanical device for movement of the piston rod 8, for example a mechanical motor for precise or prolonged dosing.

In order to prevent the piston 3 to be moved during storage or transport, the grip 12 of the piston rod 8 is secured to the finger grip 6 of the barrel 2 by a removable connector 40.

In order to prevent clogging of substance in the discharge opening 3 prior to injection, the discharge opening 3 is occluded with a closure element 5, that comprises a stern 13 extending into said discharge opening 3 for prevention of clogging of substance in the discharge opening 3. The closure element 5 is removed before connection to an intra venous access or mount of a cannula on the discharge opening 3. The closure element 5 separated from the discharge opening 3 is shown in FIG. 2.

The piston 7 can be constructed in different ways. Common for these different pistons according to the invention is that the piston 7 has means for opening a passage between the first 10 and the second chamber 11 for forcing the second substance to flow from the second chamber 11 into the first chamber 10 during retraction of said piston 7. After the retraction of the piston 7, the two substances are mixed in the, now increased, first chamber 10. Also, the piston 7 has means for closing said passage between the first 10 and the second chamber 11 for forcing the mixed substances in the first chamber 10 towards the discharge opening 3 by forward movement of said piston 7.

One embodiment of a piston is illustrated in FIG. 2 showing a syringe 1 according to the invention in side view and in longitudinal sectional view across the line A-A. Also shown in a view along the line A-A are the single elements that are mounted on the piston rod 8 and the barrel 2.

The barrel 2 in this embodiment has an oval inner cross section, which is reflected by the oval closure member 4. The closure member 4 has an air inlet 14 through the closure member 4 for air supply to said second chamber 11 during forward movement of said piston 7. Under storage of the syringe, the air inlet 14 will be closed, preferably by a sealing strip of paper or plastic, which has to be removed prior to injection of the mixed substances.

This strip may serve as an information strip identifying the medicine in the syringe 1. Upon removal of this strip, the medical personal may use this strip for documentation in the files of the patient. The strip can be provided with reusable glue making it easy to move the strip directly from the closure member 4 and into the files for the patient This way, disposal of the strip is prevented, which is an advantage. Furthermore, this feature of the syringe according to the invention increases security, because mistakes during update of the files is minimised.

In order to fill the syringe, it is advantageous if the closure member 4 is equipped with two channels 14, 14′. One channel 14 can be used for filling of the second chamber 11 with a substance, preferably liquid, while the other channel 14′ allows air to escape from the second chamber 11 during this filling. As only one of the channels 14, 14′ is used for the filling, one of the channels 14′ may be sealed permanently after filling of the second chamber 11. Alternatively both channels 14, 14′ may be sealed by a sealing strip.

The piston rod 8 extends through the closure member 4 and is sealed with a gasket, preferably an elastic o-ring 15, to prevent fluid to enter or to escape from the second chamber 11 unintentionally.

The piston of the shown syringe 1 comprises a throttle valve of mainly two parts 17 20, shown in perspective detail in the lower right part of the figure, where part 17 of the throttle valve has an oval cross section and part 20 has a circular cross section. The part 17 with oval cross section is rotatably fixed in the barrel 2, while the circular part 20 is rotatable in the barrel 2. The two parts 17 and 20 are engaged to allow opening and closing of a channel between the first 10 and the second chamber 11. The functioning of the throttle valve will be explained in the following.

Inside the barrel the first part 17 of the throttle valve piston is sealingly engaged with the inner surface of the barrel 2 by a sealing 16, preferably a o-ring like polymer gasket mounted in a groove on the periphery of the first part 17. Furthermore, the piston rod 8 extends through this first part 17 of the throttle valve in sealing conditions due to a further sealing 18, preferably an o-ring placed in a corresponding groove 170 in the first part 17. The second part 20 of the throttle valve is fastened to the piston rod 8 and, due to its circular cross section, turnable inside the barrel. The figure shows the second part 20 of the throttle valve from one side 20, which is the side facing the first part 17 of the valve, and from the opposite side 20′. When mounted on the piston rod 8, two passages will be established between the first 10 and the second chamber 11 of the syringe 1, when the channels 21 in the first part 17 of the throttle valve are aligned with the channels 22 of the second part 20 of the throttle valve. Turning the second part 20 of the throttle valve with respect to the first part 17 of the throttle valve will close or open the passages. In order to mutually seal the two parts 17, 20 such that no fluid may leak through the throttle valve, the channels 22 of the second part 20 of the throttle valve are surrounded by two sealings 19, preferably polymer o-rings, in corresponding grooves 24.

In order to facilitate operation of the throttle valve, a stem 23 on the second part 20 of the throttle valve, is engaged with a corresponding groove 25 in the first part 17 of the throttle valve. The groove defines a first and a second end stop, where the first end stop ensures closed passages between the first 10 and the second chamber 11 and the second end stop ensures open passages.

Alternatively, the barrel 2 may be of circular cross section. A possible principle is shown in FIG. 3 in a slightly simplified arrangement for illustration, where the barrel is missing and only the front end 51 of the barrel is shown. The first part 17 of the piston 4 as well as the second part, which consists of two components 20 and 20′ to increase rigidity of the piston, are circular. The first part 17 is prevented from turning due to engagement with axially extending inner ribs 50, 50′ inside the barrel (not shown) mating with a complementary groove 52 in the outer surface of said first part 17. The second part 20, 20′ is equipped with grooves 53 that allow rotation of this part 20, 20′.

In FIG. 4, a different embodiment of the piston 7 according to the invention is shown in detail. The syringe 1 comprising a barrel 2 with circular cross section has a corresponding circular closure member 4. The piston 7 in this case has a valve with two elements, a rigid disc 26 and a flexible membrane 27, mounted on the piston rod 8. The flexible membrane 27 has a cross section equal to the inner cross section of the barrel 2. The membrane 27 is mounted close to the disc 26.

When the piston 7 is retraced, which is illustrated in FIG. 5a, pressure will built up in the second chamber 11 and, due to the channels 28 in the first element 26, cause the flexible membrane 27 to deform. This deformation allows fluid to flow from the second chamber 11 through the channels 28 and around the formed peripheral passage 30 to the first chamber 10, which is illustrated with an arrow 29.

When the piston after retraction is pushed in a forward direction, which is illustrated in FIG. 5b, the flexible membrane 27 will attain its original form and rest against the rigid disc 26. The situation in the figure is just prior to injection. The air inlet in the closure member 4 is opened, and the closure element 5 is removed for connection with an intra venous access or mount of a cannula Having attained its original form, the flexible membrane 27 will engage sealingly with the inner surface of the barrel 2 and during forward push of the piston 7 cause discharge through the opened discharge opening 3.

An alternative embodiment is shown in FIG. 6. The piston, in this case consists of a rigid disc 31 sealingly fastened to the piston rod 8. The rigid disk 31 is equipped with a peripheral groove into which a flexible ring 32, preferably a polymer ring, is attached. The ring 32 has a lip 33, which in cross sectional view is illustrated in greater detail in the encircled and enlarged image, where the lip has a front plane 34 substantially normal to the axis of the barrel 2 and a back plane 35 which is conical. This form ensures that the lip 33 is easily deformed during retraction of the piston 7 in order to ensure a passage for the fluid from the second 11 to the first chamber 10. During forward push of the piston 7, however, the lip is sufficiently rigid to ensure proper discharge through the discharge opening.

A higher degree of flexibility of the flexible property of the piston during retraction and a rigid property during forward movement of the piston 7 can be achieved by a modification of the previous embodiment. In this case, the lip 33′ is enhanced in size such that the piston forms a bell shaped flexible member with a concave side towards the first chamber 10 and a convex side towards the second chamber 11. The peripheral lip of the bell shaped flexible member will bend inwards when, during retraction of said piston 7, the pressure in the first chamber 10 is lower than in said second chamber 11. This way, a passage is established between said first 10 and said second chamber 11. During forward motion, when pressure builds up in the first chamber 10 and exceeds the pressure in the second chamber 11, the lip 33′ is pressed against the inner surface of the barrel 2 and seals between the first 10 and the second chamber 11,

The invention has been illustrated with respect to medical use. However, the principle can be applied as well in connection with syringes for other chemical products, for example in food production.

Though it is the intention with the invention to provide a syringe for mixing two substances by retracting the piston first and discharging the mixture of substances after that, it is obvious that the syringe can be used for first discharging the first substance from the fist chamber, then retracting the piston to force the second substance from the second chamber into the first chamber and then discharging the second chamber. This function may be of value in case two substances, for example two glue components, are very viscous and need to be mixed manually outside the syringe.

Furthermore, it should be noted that it is not necessary to force all of the substance from the second chamber into the first chamber to be mixed with the first substance. The syringe according to the invention allows that only a controlled part of the first substance is forced into the second chamber.

1. Dual chamber syringe comprising
a hollow tubular barrel having a discharge opening at one end and a closure member at the opposite end,
a piston connected to one end of a piston rod protruding through a sealed, fluid tight opening in said closure member, wherein said piston is slidably fitted into said barrel,
wherein said volume between said piston and said discharge opening end constitutes a first chamber for a first substance,
wherein said volume between said piston and said closure member constitutes a second chamber for a second substance,
wherein said piston has means for opening a passage between said first and said second chamber for forcing said second substance to flow from said second chamber into said first chamber during retraction of said piston,
wherein said piston has means for closing said passage between said first and said second chamber for forcing said substance in said first chamber towards said discharge opening by forward movement of said piston,
wherein said closure member has means for opening an air inlet through said closure member for air supply to said second chamber during forward movement of said piston.

2. Dual chamber syringe according to claim 1, characterised in that said piston comprises a throttle valve operable to open and closing said passage by a turning of said piston rod.

3. Dual chamber syringe according to claim 1 or 2, characterised in that said hollow tubular barrel is oval or polygonal on cross section.

4. Dual chamber syringe according to claim 2, characterised in that said hollow tubular barrel has an axially extending inner rib mating with a complementary groove in the outer surface of at least one part of said piston in order to prevent that part of said piston to turn with respect to said barrel.

5. Dual chamber syringe according to claim 1, characterised in that said piston comprises a valve with a flexible component having a cross section equal to the inner cross section of said barrel, wherein said flexible component by retraction of said piston is deformable such as to allow fluid from said second chamber to flow to said first chamber.

6. Dual chamber syringe according to claim 5, characterised in that said flexible component is a membrane or a flexible ring mounted on a rigid disc.

7. Dual chamber syringe according to claim 1, characterised in that said piston comprises a bell shaped flexible member with a concave side towards said first chamber and a convex side towards said second chamber, said bell shaped flexible member having a peripheral lip arranged such as to be pressed against the inner surface of said barrel when the pressure in said first chamber is higher than in said second chamber, preferably during forward motion of said piston, in order to seal between the first and the second chamber, and arranged such as to be bend inwards when the pressure in said first chamber is lower than in said second chamber, preferably during retraction of said piston, in order to achieve said opening of said passage between said first and said second chamber.

8. Dual chamber syringe according to claim 1-7, characterised in that said discharge opening is occluded with a closure element arranged to be removed before mount of a cannula on said closure opening, where said closure opening comprises a stem extending into said discharge opening for prevention of clogging of said first substance in said discharge opening.

9. Dual chamber syringe according to claim 1-8, characterised in that said piston rod is connected to said barrel by a removable connector, said removable connector to be removed before relative movement of said piston with respect to said barrel.

10. Dual chamber syringe according to claim 1-9, characterised in that said closure member is equipped with two channels for filling of a substance into the second chamber through one of the channels and simultaneous escape of air through the second channel.
